(19) United States
(12) Patent Application Publication
(10) Pub. No.: US 2021/0236802 A1
(43) Pub. Date: Aug. 5, 2021

(54) Systems and Methods for Treating or Preventing Right and/or Left Cardiac Overload and Ventricular Disfunction

(71) Applicant: Zoll Circulation, Inc., San Jose, CA (US)

(72) Inventors: John Thomas Buckley, San Jose, CA (US); Richard Helkowski, Redwood City, CA (US); Christopher L. Kaufman, Somerville, MA (US); Gary A. Freeman, Waltham, MA (US)

(21) Appl. No.: 17/051,616

(22) PCT Filed: Apr. 29, 2019
(86) PCT No.: PCT/US2019/029732

§ 371 Date: Oct. 29, 2020

Related U.S. Application Data
(60) Provisional application No. 62/664,922, filed on Apr. 30, 2018.

(51) Int. Cl. A61M 60/531; A61M 60/38; A61M 1/14; A61M 1/36

(52) U.S. Cl. CPC A61M 60/531; A61M 60/38; A61M 1/14; A61M 2205/3327; A61M 2205/3334; A61M 2210/125; A61M 2230/30; A61M 1/3659

(57) ABSTRACT

Devices, systems and methods for controlling or preventing left and/or right ventricular overload with and without concurrent extracorporeal life support.

### RELATED APPLICATION

This PCT International Patent Application claims priority to U.S. Provisional Patent Application No. 62/664,922 filed Apr. 30, 2018 entitled Systems and Methods for Treating or Preventing Right and/or Left Cardiac Overload and Ventricular Disfunction, the entire disclosure of which is expressly incorporated herein by reference.

### FIELD

The present disclosure relates generally to the fields of medicine and engineering and more particularly to apparatus and methods for preventing cardiac disfunction and resultant deleterious cardiac and pulmonary effects.

### BACKGROUND

Pursuant to 37 CFR 1.71(e), this patent document contains material which is subject to copyright protection and the owner of this patent document reserves all copyright rights whatsoever.

In certain procedures for extracorporeal pulmonary and/or circulatory support, blood is withdrawn from a patient's vasculature, circulated through an extracorporeal blood processing or pumping system and then returned to the patient's vasculature. One example of this is Extracorporeal Membrane Oxygenation (ECMO). ECMO is a type of cardiopulmonary life-support in which blood is removed from a patient's circulatory system, circulated through an extracorporeal oxygenator which adds oxygen and removes carbon dioxide, and then infused back into the patient's circulatory system. ECMO systems have generally included venoarterial systems (“V-A ECMO”) wherein blood is removed from the patient's venous circulation and then returned into the patient's arterial circulation as well as venovenous systems (“V-V ECMO”) wherein blood is removed from and returned to the patient's venous circulation. V-A ECMO is typically used for treatment of patients suffering from cardiac failure or decreased cardiac function. V-V ECMO is typically used for treatment of patient's who suffer from respiratory failure due to lung disease or damage, acute inhalational lung injury, etc.

In traditional V-A ECMO, the venous blood is removed through a cannula positioned in a major vein and oxygenated blood is returned through a cannula positioned in a major artery, thereby completely bypassing the patient's heart and lungs. Infusion of the oxygenated blood into the arterial circulation outside of the heart can significantly increase the arterial blood pressure and can increase the pressure differential across the patient's aortic valve (i.e., the difference in pressure between blood in the aorta and blood in the left ventricle during the diastolic phase of the cardiac cycle). This is referred to as increased left ventricular afterload. Such increased cardiac afterload requires the heart's left ventricle to work harder to fully eject its entire content of blood through the aortic valve and into the aorta during the systolic phase of the cardiac cycle.

In some patients, particularly those whose heart muscle is weakened or damaged due to disease or injury, the left ventricle may not be capable of generating enough pumping force to overcome increased left ventricular afterload. This results in incomplete emptying of the left ventricle during each heartbeat. Over time, this can cause distention of the left ventricle, pulmonary hypertension and a build-up of fluid in the patient's lungs (e.g., pulmonary edema).

### SUMMARY

The present disclosure provides methods and devices for reducing or preventing right and/or left cardiac overload.

In accordance with one embodiment of the present disclosure, there is provided a system for controlled drainage of blood from a chamber of the heart of a subject to deter or reduce overload of that chamber of the heart, such system comprising: a drainage conduit positionable to drain blood from a drainage location that is within or upstream of said chamber of the heart; at least one sensor for sensing at least one parameter or variable indicative of overload of said chamber of the heart or indicative of size or dimension of the chamber of the heart or pressure within the chamber of the heart or indicative of a hemodynamic condition which causes or is associated left ventricular overload or incomplete left ventricular emptying; a flow control apparatus useable to control the amount of blood that drains through the drainage conduit; a controller that receives signals from said at least one sensor and is programmed to control, on the basis of said signals, the amount of blood being drained through the drainage conduit; and a return conduit for returning blood that has drained trough the drainage conduit into either the subject's vasculature or into another extracorporeal device through which the subject's blood is circulating. The chamber of the heart may be the left ventricle. The chamber of the heart may be the right ventricle. The flow control apparatus or flow controlling device may be selected from e.g., a valving device, a pump or a valving device and pump in combination. The drainage conduit may comprise a flexible catheter that is advanceable, transluminally, through the subject's vasculature to the drainage location, without penetrating or passing through any man made opening in the myocardium, interventricular septum or interatrial septum. The parameter(s) or variable(s) may be selected from: pressure within said chamber of the heart; afterload affecting emptying of said chamber of the heart; volume or size of said chamber of the heart; a pressure volume loop; pulse transmission rate downstream of said chamber of the heart; a difference between pressure at a first location within said chamber of the heart and pressure at a second location outside of said chamber of the heart. The drainage location may be located in or upstream of the left ventricle, and the parameter(s) or variable(s) may be indicative of left ventricular overload. The parameter(s) or variable(s) may be selected from: pressure within the left ventricle; a pressure volume loop indicating pressure/volume within the left ventricle; pulse transmission rate within the aorta (see, e.g., http://www.par-berlin.comlen/wissen/blood-pressure-and-arterial-stiffness/); extracardiac arterial pressure; aortic pressure; the difference between pressure at a first location within the left ventricle and pressure at a second location in the aorta or other extracardiac arterial vasculature. The drainage location may be in or upstream of the right ventricle, and the parameter(s) or variable(s) may be indicative of right ventricular overload. The parameter(s) or variable(s) may be selected from: pressure within the right ventricle; a pressure volume loop indicating pressure/volume within the right ventricle; pulse transmission rate within the pulmonary arterial vasculature; a pulmonary artery pressure; pulmonary artery wedge pressure; the difference between pressure at a first location within the right atrium or ventricle and a second pressure in a pulmonary artery. In some embodiments, the drainage conduit may be connected to a location in the subject's vasculature so that blood that is drained through the drainage conduit will be returned (e.g., reinfused) into the subject's vasculature. In applications where the subject is connected to another extracorporeal device through which the subject's blood is circulated (e.g., an ECMO device, a cardiopulmonary bypass device, an extracorporeal circulatory support or cardiac assist device), the return conduit may be connected to that other extracorporeal device such that blood which drains through the drainage conduit may become combined with the subject's blood circulating through the other extracorporeal device. In some applications where the other extracorporeal device has a blood inlet, the return line may be connected to the other extracorporeal device such that blood from the return conduit becomes combined with blood entering the inlet of the other extracorporeal device.

In accordance with another embodiment of this disclosure, there is provided a system for delivering blood to a subject, said system comprising an extracorporeal pulmonary and/or circulatory support system which comprises a blood inlet, a blood pump, and a blood outlet; a venous blood withdrawal cannula insertable into venous vasculature of the subject and connectable to the blood inlet; and a blood return cannula insertable into arterial vasculature of the subject and connectable to the blood outlet; a drainage conduit having a distal portion configured for insertion into the left ventricle, pulmonary vein or pulmonary artery of the subject and a proximal end connectable to the extracorporeal system such that blood may flow from the left ventricle, pulmonary vein or pulmonary artery, through the drainage conduit and into the extracorporeal system such that it combines with other blood circulating through the extracorporeal system; at least one sensor for sensing at least one parameter or variable indicative of left ventricular pressure, size or dimension or of a hemodynamic condition associated with left ventricular overload or incomplete left ventricular emptying; a flow controlling device for starting, stopping or controlling the rate at which blood will flow from the left ventricle, through the left ventricular drainage conduit and into the extracorporeal system such that it combines with other blood circulating through the extracorporeal system; and a controller which receives signals from said at least one sensor and is programmed to respond to said signals by causing the flow controlling device to start, stop, change or control the rate of blood flow from the left ventricle, pulmonary vein or pulmonary artery, through the drainage conduit and into the extracorporeal system. The flow controlling device may comprise a pump which controls the rate, if any, that blood that flows through the left ventricular drainage conduit. In some embodiments, the system may further comprise a reservoir and/or a blood oxygenator for oxygenating blood circulating through the system. The parameter(s) or variable(s) may be selected from: left ventricular pressure, left ventricular end-diastolic pressure, left ventricular end-systolic pressure, left ventricular end-diastolic volume, left ventricular end-systolic volume, pulmonary wedge pressure, cardiac afterload, pulmonary vein pressure and the difference between left ventricular pressure and extracardiac arterial pressure. In systems wherein left ventricular pressure is a sensed parameter or variable, the drainage conduit may be placed in the left ventricle and the sensor(s) may comprise pressure sensor(s) located on the drainage conduit so as to directly measure left ventricular pressure. In systems where pulmonary wedge pressure is a sensed parameter or variable, the drainage conduit may be placed in the pulmonary vasculature. The sensor(s) may comprise a pressure sensor and balloon useable in combination to measure pulmonary wedge pressure. In systems where pulmonary venous pressure is a sensed parameter or variable, the drainage conduit may be placed in a pulmonary vein. The sensor(s) may comprise a pressure sensor located on the drainage conduit so as to measure said pulmonary venous pressure. The sensed variable or parameter may comprise a difference between a first pressure measured at a first location selected from the left ventricle, pulmonary vein or pulmonary artery and a second location in the aorta or extracardiac arterial vasculature. The drainage conduit may be placed at the first location. The at least one sensor may comprise a first pressure sensor for sensing pressure at the first location and a second sensor for sensing pressure at the second location. The sensor may be located on a separate pulmonary artery wedge pressure measuring catheter that is useable to measure pulmonary artery wedge pressure. The controller may receive signals from the sensor indicating pulmonary artery wedge pressure and the controller may be programmed to correlate pulmonary artery wedge pressure to left ventricular pressure. The flow controlling device may comprise a valving device which controls the amount of blood that flows through the left ventricular drainage conduit. A blood pump of the extracorporeal system may draw blood through both the venous blood withdrawal cannula and the left ventricular drainage conduit. The valving device may vary the relative amounts of blood being drawn through the venous blood withdrawal cannula and left ventricular drainage conduit. The flow controlling device may comprise a pump which controls the rate, if any, that blood flows through the left ventricular drainage conduit. The pump may be separate from the blood pump of the extracorporeal system or may be a pump within the extracorporeal system. The flow controlling device may comprise a valving device and a pump, in combination. In other systems, the blood pump of the extracorporeal system may provide pumping force to move blood through the drainage conduit and through the extracorporeal system. The drainage conduit may comprise a first lumen of a catheter device. The blood return cannula may comprise a second lumen of that same catheter device. The catheter device may comprise a first tube having a first lumen which may function as the drainage conduit. The catheter device may have a second tube having a second lumen which may function as the blood return cannula. The second tube may be initially positionable within the aorta and the first tube may be subsequently advanceable from the second tube, through the aorta, across the aortic valve and into the left ventricle. Such second tube may include a utility lumen in addition to the second lumen. The first tube may be advanceable through the utility lumen of the second tube. The first lumen may communicate with a left ventricular blood withdrawal opening or port located in a distal portion of the catheter device. The second lumen may communicate with a blood infusion opening or port located on the catheter device proximal to the left ventricular blood withdrawal opening or port. The first lumen may be connectable to the blood inlet of the extracorporeal system. The second lumen may be connectable to the blood outlet of the extracorporeal system. The venous blood withdrawal cannula may also be connected to the blood inlet of the extracorporeal system. The blood from the venous blood withdrawal cannula and the drainage conduit may become combined in or ahead of the blood inlet of the extracorporeal system. In some embodiments, the controller may be programmed or otherwise configured to ratiometrically or otherwise change or adjust the amounts of blood entering the blood inlet from the drainage conduit and venous blood withdrawal conduit, respectively. The pump of the extracorporeal system may create sufficient extracardiac arterial pressure to adequately perfuse the patient's organs and tissue. The controller may be programmed to maintain pressure within the left ventricle below the arterial pressure, e.g., below a pressure that would cause the aortic valve to open and ejection of blood from the left ventricle into the aorta during systolic contraction of the heart.

In accordance with another embodiment of the present disclosure, there is provided a shunt device for shunting blood from the left ventricle in a subject who is in need thereof and who has a heart, left ventricle, aorta, aortic valve, arterial vasculature and venous vasculature, said device comprising: a conduit having a first end configured for insertion into the subject's arterial vasculature and advancement through the aorta, through the aortic valve and into the left ventricle and a second end that is insertable into the subject's venous vasculature so that flood will flow from the left ventricle, through the conduit and into the subject's venous vasculature. Such shunt may further comprise a pump which pumps blood from the left ventricle, through the conduit and into the venous vasculature. Such shunt device may further comprise a controller that receives signals indicative of a parameter or variable that indicates when the subject would benefit from removal of blood from the left ventricle. The controller may be programmed to control said pump in response to the received signals such that the pump will pump blood from the left ventricle through the conduit and into the venous vasculature to provide a benefit to the subject. The parameter(s) or variable(s) may be selected from: left ventricular pressure, left ventricular end-diastolic pressure, left ventricular end-systolic pressure, left ventricular end-diastolic volume, left ventricular end-systolic volume, pulmonary wedge pressure and cardiac afterload. Such shunt device may further comprise valving apparatus which controls when blood may vent from the left ventricle, through the conduit and into the venous vasculature. The device may further comprise a controller that receives signals indicative of a parameter or variable that indicates when the subject would benefit from allowing blood to vent from the left ventricle through the conduit and into the venous vasculature. The controller may be programmed to control when said valving device allows blood to vent from the left ventricle, through the conduit and into the venous vasculature. Such parameter(s) or variable(s) may be selected from left ventricular pressure, left ventricular end-diastolic pressure, left ventricular end-systolic pressure, left ventricular end-diastolic volume, left ventricular end-systolic volume, pulmonary wedge pressure and cardiac afterload.

In accordance with another embodiment of the present disclosure, there is provided a system for delivering oxygenated blood to a subject, such system comprising: an extracorporeal blood oxygenating system having a blood inlet, a blood pump, a blood oxygenator and a blood outlet; a venous blood withdrawal cannula insertable into venous vasculature of the subject and connectable to the blood inlet; and an oxygenated blood return cannula insertable into arterial vasculature of the subject and connectable to the blood outlet; a drainage conduit having a distal portion configured for insertion into the left ventricle, pulmonary vein or pulmonary artery of the subject and a proximal end connectable to the extracorporeal blood oxygenating system such that blood may flow from the left ventricle, pulmonary vein or pulmonary artery, through the drainage conduit and into the extracorporeal blood oxygenating system such that it combines with other blood circulating through the extracorporeal blood oxygenating system; at least one sensor for sensing at least one parameter or variable that indicates when the subject would benefit from removal of blood from the left ventricle; and a controller which receives signals from said at least one sensor and is programmed to respond to said signals starting, stopping or varying the rate of blood flow from the left ventricle, pulmonary vein or pulmonary artery, through the drainage conduit and into the extracorporeal blood oxygenating system such that it combines with other blood circulating through the extracorporeal blood oxygenating system. The system may comprise a reservoir.

In accordance with another embodiment of the present disclosure, there is provided a left ventricular unloading system useable in conjunction with an extracorporeal system for pulmonary and/or circulatory support of a subject. Such system comprises a drainage and pressure sensing cannula having a drainage lumen, a first pressure sensor and a second pressure sensor, said drainage and pressure sensing cannula being insertable to an operative position in the subject's body wherein i) the first pressure sensor is positioned in a left ventricle, ii) the second pressure sensor is positioned in an aorta and iii) blood will drain from the left ventricle though the drainage lumen, said drainage lumen being connectable to the extracorporeal system for pulmonary and/or circulatory support such that blood which drains from the left ventricle though the drainage lumen will become combined with blood circulating through the extracorporeal system for pulmonary and/or circulatory support; a flow control device for controlling the rate, if any, at which blood flows through the draining lumen; and a controller which receives pressure indicating signals from the first and second pressure sensors and is programmed to use those pressure indicating signals received from the first and second pressure sensors to control the flow control device to adjust as necessary the rate at which blood flows from the left ventricle, through the drainage lumen and into the extracorporeal system for pulmonary and/or circulatory support. The flow control device may comprise a pump, a valving apparatus or a pump and valving apparatus in combination. In systems where the flow control apparatus comprises a pump, the controller may be programmed to determine the difference between the left ventricular pressure sensed by the first pressure sensor and the aortic pressure sensed by the second pressure sensor. The controller may be configured to compare that difference to a preset acceptable difference value or range or user-input acceptable difference value or range. If the current difference is equal to an acceptable difference value or within an acceptable difference range, the controller may be configured to make no change in the rate, if any, at which the pump is pumping blood from the left ventricle, through the drainage lumen and into the extracorporeal system for pulmonary and/or circulatory support. If the current difference is not equal to the acceptable difference value or within an acceptable difference range, the controller may be configured to start, stop or change the rate at which the pump is pumping blood from the left ventricle, through the drainage lumen and into the extracorporeal system for pulmonary and/or circulatory support to cause the difference to subsequently become equal to the acceptable difference value or within an acceptable difference range.

In accordance with another embodiment of the present disclosure, there is provided a system for delivering oxygenated blood to a subject, such system comprising: an extracorporeal blood oxygenating system which includes a blood inlet, a blood pump, a blood oxygenator, and a blood outlet. The system comprises a venous blood withdrawal cannula insertable into venous vasculature of the subject and connectable to the blood inlet; an oxygenated blood return cannula insertable into arterial vasculature of the subject and connectable to the blood outlet; a drainage conduit having a distal portion configured for insertion into the left ventricle, pulmonary vein or pulmonary artery of the subject and a proximal end connectable to the extracorporeal blood oxygenating system such that blood may flow from the left ventricle, pulmonary vein or pulmonary artery, through the drainage conduit and into the extracorporeal blood oxygenating system such that it combines with other blood circulating through the extracorporeal blood oxygenating system; at least one sensor for sensing at least one variable indicative of left ventricular pressure, size or dimension or of a hemodynamic condition which causes or is associated with left ventricular overload or incomplete left ventricular emptying; a flow controlling device for controlling the rate, if any, at which blood will flow from the left ventricle, through the left ventricular drainage conduit and into the extracorporeal blood oxygenating system such that it combines with other blood circulating through the extracorporeal blood oxygenating system; and a controller which receives signals from said at least one sensor and is programmed to respond to said signals by causing the flow controlling device to start, stop or vary the rate of blood flow from the left ventricle, pulmonary vein or pulmonary artery, through the drainage conduit and into the extracorporeal blood oxygenating system such that it combines with other blood circulating through the extracorporeal blood oxygenating system. The system may comprise a reservoir.

In accordance with another embodiment of the present disclosure, there is provided a left ventricular unloading system useable in conjunction with an extracorporeal system for pulmonary and/or circulatory support of a subject, such left ventricular unloading system comprising: a drainage and left ventricular pressure and/or volume determining cannula having a drainage lumen and a pressure and/or volume sensing apparatus useable to determine the size and/or volume of the left ventricle, said cannula being insertable to an operative position in the subject's body wherein i) the pressure and/or volume sensing apparatus is positioned to measure the size and/or volume of the left ventricle and ii) blood will drain from the left ventricle in a proximal direction though the drainage lumen, said drainage lumen being connectable to the extracorporeal system for pulmonary and/or circulatory support such that blood which drains from the left ventricle in the proximal direction though the drainage lumen will become combined with blood circulating through the extracorporeal system for pulmonary and/or circulatory support; a flow control device useable for controlling the rate, if any, at which blood flows in the proximal direction through the draining lumen; and a controller which receives left ventricular pressure and/or volume signals from the left ventricular pressure and/or volume sensing apparatus and is programmed to use those signals to adjust the rate at which blood flows from the left ventricle, through the drainage lumen and into the extracorporeal system for pulmonary and/or circulatory support as necessary to deter the left ventricular pressure and/or volume, or a derivative thereof, from exceeding a limit. The left ventricular pressure and/or volume sensing apparatus may sense left ventricular volume by conductance or impedance. The left ventricular pressure and/or volume sensing apparatus may provide electrical conductance signals to the controller. The controller may be programmed to use those signals to calculate left ventricle size. The left ventricular pressure and/or volume sensing apparatus may provide, to the controller, signals representing both pressure and volume information. The controller may be programmed to compute a left ventricular pressure-volume loop and then issue control signals to the flow control device based on the computed pressure-volume loop. The pressure and/or volume sensing apparatus may sense/measure both pressure and conductance and may transmit pressure and conductance signals to the controller. The controller may be programmed to use the received pressure and conductance signals to compute a left ventricular pressure-volume loop and to determine whether one or more parameters of the computed pressure-volume loop is/are acceptable or not acceptable.

In accordance with yet another embodiment of the present disclosure, there are provided methods for using devices and systems of the type disclosed herein. In one embodiment, there is provided a method for deterring left ventricular overload in a subject who is being treated by an extracorporeal circulatory support system wherein blood is withdrawn through a venous cannula from venous vasculature of the subject, and returned through an arterial cannula into the extracardiac arterial vasculature of the subject; said method comprising the steps of: positioning a drainage catheter in a left ventricle, pulmonary artery or pulmonary vein of the subject; connecting the drainage conduit to the extracorporeal circulatory support system; placing at least one sensor in the subject body to monitor at least one parameter or variable indicative of left ventricular pressure, size or dimension or of a hemodynamic condition associated with left ventricular overload or incomplete left ventricular emptying; and starting, stopping or varying/changing the amount of blood flowing through the drainage conduit in response to signals received from the sensor. The parameter(s) or variable(s) may be selected from: left ventricular pressure, left ventricular end-diastolic pressure, left ventricular end-systolic pressure, left ventricular end-diastolic volume, left ventricular end-systolic volume, pulmonary wedge pressure, cardiac afterload, pulmonary vein pressure and the difference between left ventricular pressure and extracardiac arterial pressure. In methods where left ventricular pressure is a sensed parameter or variable, the drainage conduit may be placed in the left ventricle and the sensor(s) may comprise a pressure sensor located on the drainage conduit so as to directly measure the left ventricular pressure. In methods where pulmonary wedge pressure is a sensed parameter or variable, the drainage conduit may be placed in the pulmonary artery and the sensor(s) may comprise a pressure sensor and balloon useable in combination to measure pulmonary wedge pressure. In methods where pulmonary venous pressure is a sensed parameter or variable, the drainage conduit may be placed in a pulmonary vein and the sensor(s) may comprise a pressure sensor located on the drainage conduit so as to measure said pulmonary venous pressure. In some methods, the parameter(s) or variable(s) may comprise a difference between a first pressure measured at a location (e.g., the left ventricle, pulmonary vein or pulmonary artery) and a second pressure measured at another location (e.g., the aorta or extracardiac arterial vasculature). The drainage conduit may be placed at the first location and the sensor(s) may comprise a first pressure sensor for sensing pressure at the first location and a second sensor for sensing pressure at the second location. The extracorporeal circulatory support system may create sufficient extracardiac arterial pressure to adequately perfuse the patient's organs and tissue. Blood flow through the drainage conduit may be started, stopped or varied as necessary to maintain pressure within the left ventricle below that which would cause the aortic valve to open and ejection of blood from the left ventricle into the aorta during systolic contraction of the heart. The flow of blood through the drainage conduit may be started, stopped or varied as needed to maintain pressure within the left ventricle below 80 mm/Hg or at least 20 mm/Hg below pressure in the aorta.

In certain embodiments described herein, the flow control apparatus or flow controlling device may be selected from, e.g., a valving device, a pump or a valving device and pump in combination. In embodiments wherein the flow control apparatus or flow controlling device comprises a pump, the pump may be of any suitable type, including for example centrifugal pump and positive displacement pumps such as peristaltic pumps, diaphragm pumps, gear pumps, screw pumps, rotary vein pumps, piston pumps, circumferential piston pumps, plunger pumps, etc. In embodiments where the flow control apparatus or flow controlling device comprises a valving device, such valving device may comprise any suitable type of valving device including, for example, valves, needle valves, fixed flow control valves, toggle valves, solenoid valves, toggle valves, metered flow control valves, ball valves, bull port valves, globe valves, duckbill valves, stopcocks, clamps, clips, obturators, flow-blocking members, conduit compressors, conduit constrictors, etc.

In certain embodiments described herein, the system includes an ECMO device or blood oxygenator. However, optionally the ECMO device or blood oxygenator may be absent, or another type of intracorporeal or extracorporeal blood treatment device or a circulatory support or assistance device may be substituted for, or added in addition to the ECMO device or blood oxygenator. Examples of other types of intracorporeal or extracorporeal blood treatment or circulatory support or assistance devices that may be substituted for or added in addition to the ECMO device or blood oxygenator include, but are not necessarily limited to; blood cleansing or purifying devices, blood pumping devices, blood warming or cooling devices, carbon dioxide removal devices, autotransfusion devices, hemofiltration devices, hemodialysis devices, apheresis devices, plasmapheresis devices; photophoresis devices, antimicrobial devices and gene therapy devices. Intracorporeal or extracorporeal blood treatment or circulatory support or assistance devices may or may not provide oxygenation of blood.

Also, in certain embodiments described herein, the system includes a reservoir; however, optionally the reservoir may be absent.

Still further aspects and details of the present disclosure will be understood upon reading of the detailed description and examples set forth herebelow.

### BRIEF DESCRIPTION OF THE DRAWINGS

The following detailed description and examples are provided for the purpose of non-exhaustively describing some, but not necessarily all, examples or embodiments of the disclosure, and shall not limit the scope of the disclosure in any way.

FIG. 1 shows an example of a V-A ECMO system of the prior art.

FIG. 2 shows one example of an ECMO system.

FIG. 2A is a cross sectional view through Line 2A-2A of FIG. 2.

FIG. 2B is a partial view of a distal portion of an aortic/left ventricular cannula component of the system of FIG. 2.

FIG. 2C is a partial view of a distal portion of an aortic/left ventricular cannula component of the system of FIG. 2 with an alternative curled or “pigtail” configuration.

FIG. 3 shows another example of an ECMO system.

FIG. 3A is a cross sectional view through Line 3A-3A of FIG. 3.

FIG. 3B is a partial view of a distal portion of an aortic/left ventricular cannula component of the system of FIG. 3.

FIG. 4 shows another embodiment of an ECMO system.

FIG. 5 shows another embodiment of an ECMO system.

FIG. 5A is a cross sectional view through Line 5A-5A of FIG. 5.

FIG. 5b is a partial view of a distal portion of a convertible aortic/left ventricular cannula component of the system of FIG. 5.

FIG. 6 shows another embodiment of an ECMO system.

FIG. 7 shows one embodiment of a system which uses a pressure sensing and venting catheter to unload the left ventricle during treatment with a separate ECMO system.

FIG. 7A is a cross sectional view through Line 7A-7A of FIG. 7.

FIG. 7B shows use of the system of FIG. 7 with alternative placement of the pressure sensing and venting catheter in the pulmonary artery.

FIG. 8 shows a system useable in conjunction with a Swan Ganz pulmonary artery catheter to effect controlled drainage of blood from the left ventricle.

FIG. 9 shows an alternative placement of a drainage cannula in a pulmonary vein.

FIG. 10 shows an example of a left heat unloading system which uses a difference or delta between left ventricular pressure and aortic pressure for feedback control of the amount of blood being removed from the left ventricle.

FIG. 10A is a cross-sectional view through Line 10A-10A of FIG. 10.

FIG. 10B is a general diagram of controller logic used by the system of FIG. 10.

FIG. 11 shows one embodiment of a system for unloading the right heart.

FIG. 12 shows another embodiment of a system for unloading the right heart.

### DETAILED DESCRIPTION

The following detailed description and the accompanying drawings to which it refers are intended to describe some, but not necessarily all, examples or embodiments of the disclosure. The described embodiments are to be considered in all respects only as illustrative and not restrictive. The contents of this detailed description and the accompanying drawings do not limit the scope of the disclosure in any way.

Although the accompanying drawings and examples described herein may refer to an extracorporeal oxygenation or ECMO system, it is to be understood that these are non-limiting examples. The systems and methods described herein may be used in conjunction with or included in any system which infuses blood into the extracardiac arterial vasculature or may otherwise increase cardiac afterload in a manner that renders the patient at risk for left ventricular overload, distention and/or disfunction. These include circulatory support or assistance devices, for example intracorporeal or extracorporeal circulatory support or assistance devices that pump but do not oxygenate blood or those that pump and do oxygenate blood.

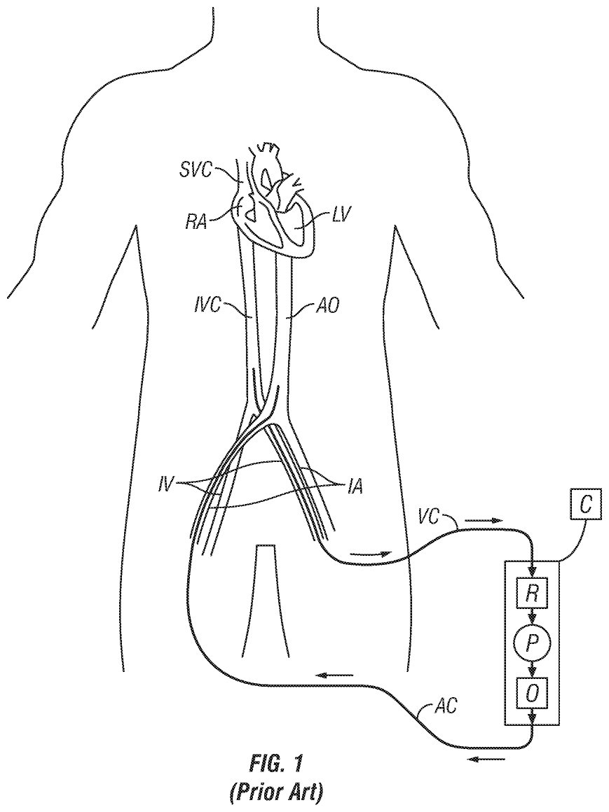

FIG. 1 shows an example of a V-A ECMO system. In this example, a venous cannula (e.g., a catheter or other suitable conduit) VC is percutaneously inserted into a femoral vein and advanced through the iliac vein IV to a position within the subject's inferior vena cava IVC. An arterial cannula (e.g., a catheter or other suitable conduit) AC is percutaneously inserted into a femoral artery and advanced through the iliac artery IA to a position within the subject's aorta AO. Various alternative insertion points and cannula positioning may also be used. The venous cannula VC is connected to the inlet of an ECMO system 12 which comprises a reservoir R, a pump P and an oxygenator O. Optionally, the ECMO system 12 may also include a controller C that is programed and operable to control the operation of the ECMO system 12. As used herein the term “controller” shall be inclusive of any electronic control apparatus or circuitry that is programmed or programmable to perform the intended functions, including but not limited to programmable logic controllers (PLCs), microprocessors, computers or other control devices as known in the art for performing the functions described herein. In embodiments where a controller receives a setting or other information input, such controller may incorporate or be connected to a user interface that is useable to input such setting or other information, such as a touch screen display, dial(s), button(s), knob(s), switch(es), etc.

The arterial cannula AC is connected to the outlet of that ECMO system, as shown. In operation, venous blood flows into the reservoir R and is pumped by pump P through oxygenator O and then through arterial cannula AC and into the subject's aorta AO. Carbon dioxide is removed from, and oxygen is added to, the blood as it passes through the oxygenator O. Thus, venous blood is removed from the subject's inferior vena cava IVC and oxygenated blood is delivered directly into the subject's aorta AO. In this manner, the ECMO circuit essentially bypasses the subject's heart and diminishes the volume of blood that is pumped through the heart while the ECMO procedure is ongoing. Also, the delivery of oxygenated blood into the aorta AO, or alternatively elsewhere in the arterial vasculature outside the heart, causes an increase in arterial blood pressure and increased cardiac afterload, which may result in left ventricular overload and distention. In order to overcome this increased cardiac afterload, the subject's heart must pump blood to a higher pressure in order to eject blood from the left ventricle LV into the aorta AO. If the heart fails to generate the requisite pressure to fully eject blood from the left ventricle LV into the aorta AO, the left ventricle LV will not empty completely. This can result in various sequelae such as distention of the left ventricle LV, development of pulmonary hypertension and pulmonary edema resulting in respiratory failure. The present disclosure describes new and improved methods and devices for left ventricular unloading, e.g., lowering the pressure or reducing the volume in the left ventricle, or otherwise preventing the deleterious effects of increased cardiac afterload and left ventricular overload and distension in patients undergoing ECMO or circulatory support therapy.

### Two Cannula LV to a System

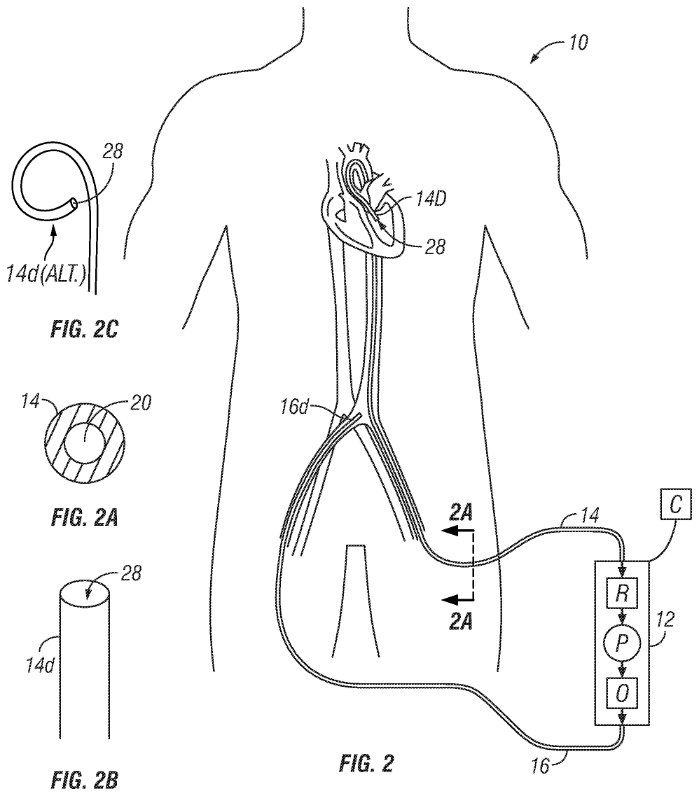

FIGS. 2 through 2B show a system 10 in which a left ventricular cannula or drainage conduit 14 has a distal portion 14d which is insertable into the subject's left ventricle LV. The proximal end of cannula 14 is connected to the inlet of an ECMO device 12. A return cannula 16 has a distal portion 16d positioned in the subject's aorta AO or other suitable location in the arterial vasculature outside of the subject's heart (i.e., extracardiac arterial location) and is connected to the outlet of the ECMO device 12. The ECMO device 12 may include a reservoir R, pump P and oxygenator O, and may optionally include a controller C and various other components. While a reservoir R is shown in FIG. 2, optionally, the ECMO device may not include a reservoir R in various embodiments. Also, in FIGS. 2 through 2B as well as any all other embodiments described herein in which an ECMO device or blood oxygenator is included, the present disclosure shall be understood to include alternative embodiments wherein the ECMO device or blood oxygenator is absent, or wherein another type of intracorporeal or extracorporeal blood treatment device or a circulatory support or assistance device is substituted for, or added in addition to, the ECMO device or blood oxygenator. Examples of other types of intracorporeal or extracorporeal blood treatment or circulatory support or assistance devices that may be substituted for or added in addition to the ECMO device or blood oxygenator include, but are not necessarily limited to; blood cleansing or purifying devices, blood pumping devices, blood warming or cooling devices, carbon dioxide removal devices, autotransfusion devices, hemofiltration devices, hemodialysis devices, apheresis devices, plasmapheresis devices; photophoresis devices, antimicrobial devices and gene therapy devices. Intracorporeal or extracorporeal blood treatment or circulatory support or assistance devices may or may not oxygenate blood.

Also, in FIGS. 2 through 2B as well as any all other embodiments described herein in which a system includes a reservoir R, the present disclosure shall be understood to include alternative embodiments wherein the reservoir R is absent.

The left ventricular cannula 14 has a lumen 20 which extends to an opening at the distal end of the left ventricular cannula 14. The distal portion 14d of this cannula 14 may be configured and constructed to facilitate its transaortic advancement through the aortic valve AV and into the left ventricle LV. For example, this left ventricular cannula 14 may have an atraumatic tip or non-atraumatic tip, which may have a straight, coiled, curled, helical or pigtail configuration for advancement into the left ventricle via the aorta or other vessel. Examples of such catheters include angiography catheters, intended for left ventricular insertion. One example of a pigtail configuration that may be used in this left ventricular cannula may include a pigtail that extends distally from the distal end of the cannula. In one example, the pigtail may be about two centimeters or less in diameter and/or have a curved or spiral coil shape extending around approximately 360 degrees or less. FIG. 2C shoes an alternative distal portion 14d (alt) of the cannula 14 having a curled or pigtail configuration. The pigtail may be sufficiently flexible to be straightened by the passage of a guide wire to allow the pigtail to be passed through the aortic valve and into the left ventricle. Once in the left ventricle, the pigtail may resume its shape upon removal of the guidewire. The pigtail may serve to anchor the distal portion within the left ventricle and/or present a blunt rounded structure to the internal ventricular wall to reduce the risk of trauma to the wall. One commercially available pigtail-type catheter is the 7-French Pigtail Catheter (Ventricular Pigtail Catheter 527-750 7 PIG 110 cm 12SH) available from Cordis Corporation, Johnson & Johnson Health Care Systems, Piscataway. N.J.) In operation of the system 10, blood is pumped by pump P from the left ventricle LV, through lumen 20, into reservoir R and through oxygenator O wherein carbon dioxide is removed and oxygen is added (or from lumen 20 to the oxygenator where no reservoir is present). The oxygenated blood is then delivered through return cannula 16 into the subject's extracardiac arterial vasculature. The pump P may withdraw blood from the left ventricle LV at a rate that causes the left ventricle LV to remain sufficiently empty, e.g., so as not to cause the aortic valve AV to open during systolic contraction of the heart. The returning of oxygenated blood may be pumped through the return cannula 16 at a rate that creates adequate arterial blood pressure within the subject's extracardiac arterial vasculature to perfuse the subject's organs and tissues.

Alternatively, the cannula 14 may be used separately from an ECMO system to unload the left heart, remove blood from LV (or pulmonary vein to bypass the LV) and deliver removed blood into the aorta. In such application the cannula 14 would have a smaller diameter than an ECMO venous cannula and the purpose of this cannula would be to unload the left heart.

In the non-limiting example shown in FIG. 2, the left ventricular cannula 14 has been percutaneously inserted via the left femoral artery and advanced through the aorta AO, across the aortic valve AV and into the left ventricle. The return cannula 16 has been percutaneously inserted via the right femoral artery to a position in the aorta AO near the iliac bifurcation. However, as those of skill in the art will readily understand, other insertion points and routes of insertion, as well as other extracardiac intra-arterial positions for the distal portion 16d of the return cannula 16 may alternatively be employed.

### Single Cannula LV to a System

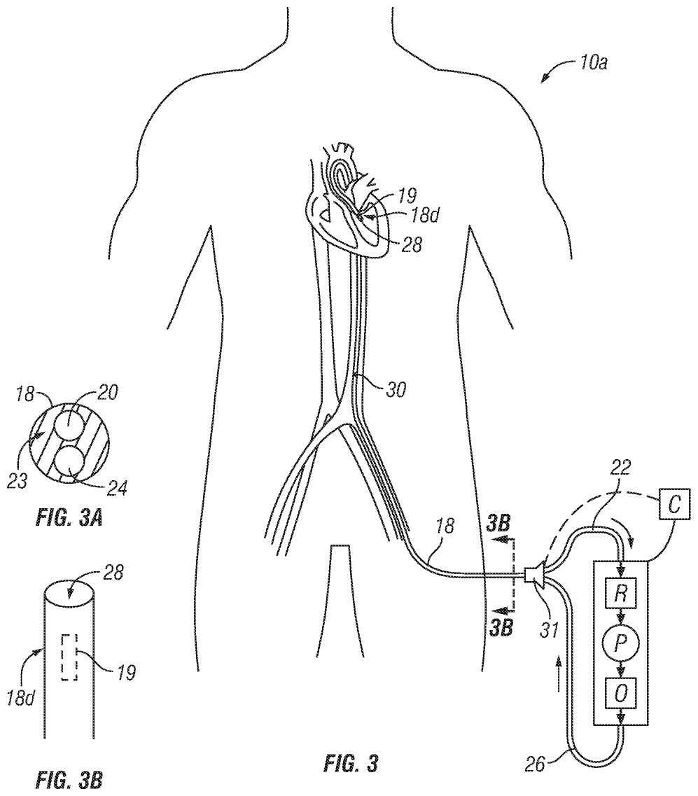

FIGS. 3A through 38 show a system 10a wherein a single cannula 18 having dual lumens 20, 24 is inserted through the aorta AO to a position where a distal portion 18d of the cannula 18 is within the left ventricle LV. A first lumen 20 extends from a distal port or opening located in the distal portion 18d to a proximal port on the hub 31, which is connected via conduit 22 to the inlet of the ECMO system 12. A second lumen 24 extends from a port or opening 30 on a portion of the catheter 18 that resides in the aorta AO and extends to a proximal port on the hub 31, which is connected by conduit 26 to the outlet of the ECMO device 12. Blood is drawn from the left ventricle LV through the first lumen 20 and oxygenated blood is returned through the second lumen 24, out of port or opening 30 and into the aorta AO. FIGS. 3A-36 also show other optional components that may be incorporated into any of the systems disclosed herein, wherever applicable, including in the system of FIG. 2. Such optional components comprise at least one optional sensor 19 positioned on the distal portion of the cannula 18 to sense pressure and/or flow within the left ventricle LV. Sensor(s) 19 communicate via a wire 23, or alternatively by wireless connection, to an optional controller C. The controller C receives signals from the sensor(s) 19 and is programmed to control the speed or other operational aspects of the pump P, and/or other aspects of the ECMO device 12, to maintain the pressure or flow rate within the left ventricle LV above or below a particular limit or within a particular range. For example, the controller C may be programmed to cause the pump P to withdraw blood from the left ventricle LV e.g., at a rate that causes the left ventricle LV to remain sufficiently empty so as not to cause the aortic valve AV to open during systolic contraction of the heart, and the returning of oxygenated blood will be pumped through the second cannula at a rate that creates adequate arterial blood pressure within the subject's arterial vessels to perfuse the subject's organs and tissues. For example, the controller may be programed to maintain a pressure within the left ventricle at some value below 80 mmHg, optionally the controller may be programmed to prevent LV blood pressure from exceeding 20 mmHg.

### V+LV to a System

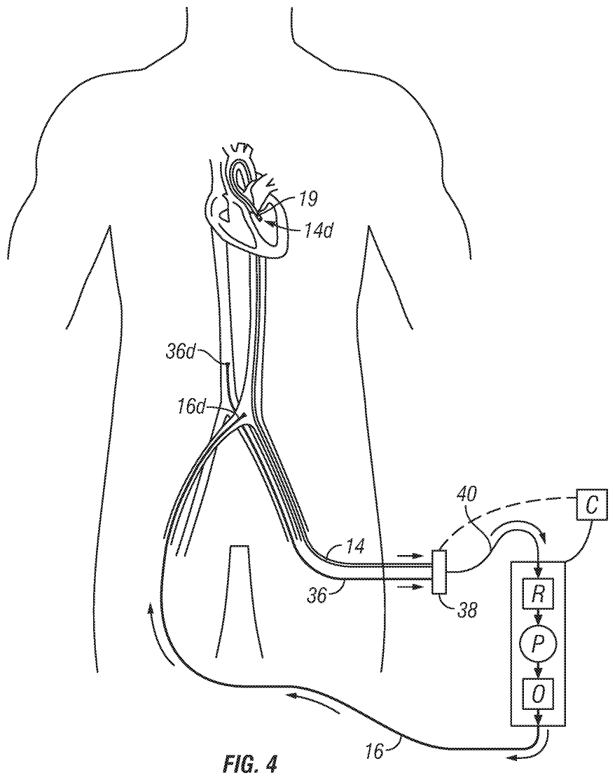

FIG. 4 shows a system 10b in which blood is drawn from both the venous vasculature and left ventricle LV and oxygenated blood is returned into the aorta or other extracardiac arterial vasculature. In this system 10b a left ventricular cannula or drainage cannula 14 of the type shown in FIG. 2 and described above, is advanced through the aorta AO and to a position where its distal portion 14d is within the left ventricle LV. A venous cannula 36 is positioned in the subject's inferior vena cava IVC or at some other suitable location in the subject's venous vasculature. The proximal ends of the left ventricular cannula 14 and venous cannula 36 are connected to a flow controlling device 38 which may, in some embodiments, be a Y connector or manifold and in other embodiments may be an adjustable valve which is adjustable to vary the relative amounts of blood being drawn through the left ventricular cannula 14 and venous cannula 36. The combined flows from left ventricular cannula 14 and venous cannula 36 are delivered from flow controlling device 38 through inflow conduit 40 to the blood inlet of the ECMO device 12. Alternatively, left ventricular cannula or drainage cannula 14 and/or venous cannula 36 may connect directly to blood inlets of the ECMO device 12. Separate adjustable valves to control the amounts of blood being drawn through or rate of blood flow through each cannula may be utilized. The flow controlling device may control the rate at which blood flows through one or more of the cannulas.

Return cannula 16 of the type shown in FIG. 2 and described above is connected to the oxygenated blood outlet of the ECMO device 12 and is inserted into the subject's arterial vasculature so that its distal portion 16d is positioned in the subject's aorta AO or at some other suitable extracardiac arterial location.

In operation, left ventricular blood and venous blood are drawn by pump P, through left ventricular cannula 14 and venous cannula 36 respectively, then through flow controlling device 38, through inflow conduit 40 and into reservoir R. The blood is then further pumped by pump P from reservoir R through oxygenator O, through return cannula 16 and into the subject's aorta AO or other suitable location in the extracardiac arterial vasculature. While a reservoir is shown in FIG. 4, optionally, the ECMO device may include no reservoir R. Also, as explained above, instead of an ECMO device 12, the cannulas may be connected to a circulatory support or assistance device, for example intracorporeal or extracorporeal circulatory support or assistance devices that have the same components described herein without the oxygenator, and which devices pump but do not oxygenate blood.

As described above, the left ventricular catheter 14 may optionally be equipped with one or more sensor(s) 19 for sensing pressure and/or flow within the left ventricle LV. Also, the flow controlling device 38 may optionally comprise an adjustable valve. The optional controller C may be programmed to receive signals from such sensor(s) 19 and to control the flow controlling device 38 alone, or to control both the flow controlling device 38 and pump P and/or other aspects of the ECMO device 12, to adjust the flow rate and/or relative amount of blood being withdrawn from the left ventricle LV as needed to maintain the sensed pressure or flow rate in the left ventricle below a particular limit or within a particular range. For example, if the pressure within the left ventricle LV exceeds an upper limit, the controller C may adjust the flow controlling device 38 to change the ratio of left ventricle blood to venous blood being drawn from the subject to prevent left ventricular overload and its resultant deleterious effects. In certain embodiments, valves or other flow control mechanisms may be located elsewhere in the system, e.g., in the cannulas, inflows conduits, blood inlets or other components, e.g., where a flow controlling device 38 may not be utilized.

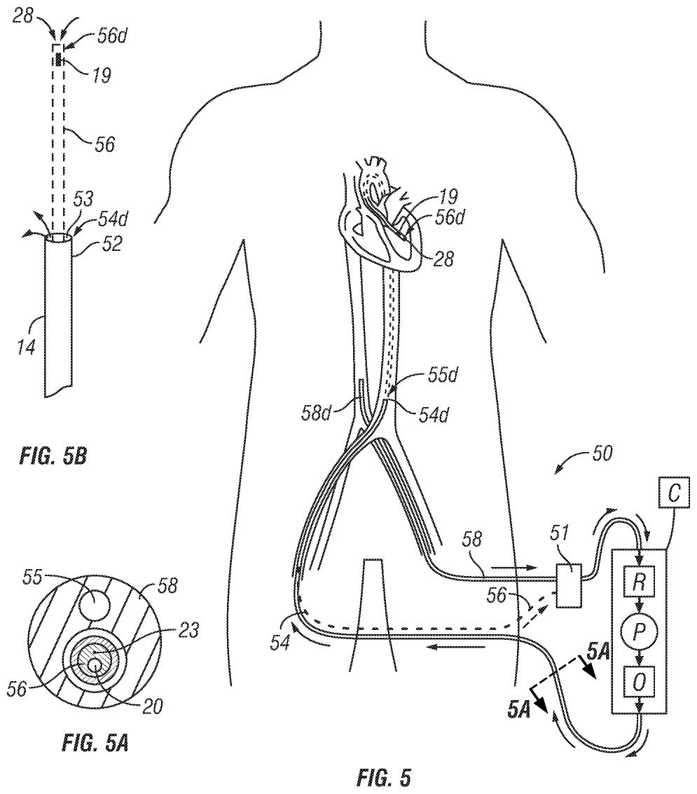

FIGS. 5A through 5B show a system 10c in which a convertible aortic return/left ventricular venting cannula 55 is inserted to a position where its distal portion 55d is in the subject's aorta AO and a withdrawal cannula 58 is inserted so that its distal portion 58d is positioned in the subject's inferior vena cava IVC or other suitable venous withdrawal location. The convertible aortic return/left ventricular venting cannula 55 has a return lumen 59 which extends from a first port or opening 52 in the distal end 55d of the cannula 55 to a proximal end of the cannula 55 where it is connected to a combining member 38 of the type described above. The aortic return/left ventricular venting cannula 55 also has a second lumen 60 which extends from a second opening or port 54d in the distal end 55d to a side opening or port 61 located in a proximal portion of the cannula that remains exteriorized during use. A left ventricular venting cannula 56 having distal end 56d is insertable through side opening or port 61 and advanceable through the second lumen 60. As those of skill in the art readily understand, a hemostatic device such as a Tuohy Borst adapter may be positioned on the side port or opening 61 to deter leakage of blood around the left ventricular venting cannula 56 during use. The left ventricular venting cannula 56 may have substantially the same configuration and lumen 20 as the left ventricular cannula 14 described above in relation to FIG. 2. Additionally, in the embodiment shown, the left ventricular venting cannula 56 may include an optional sensor 19 and sensor wire 18, as described above in relation to FIG. 3 for sensing various patient parameters and providing feedback to a controller.

At the time of initial insertion, the left ventricular venting catheter 56 may not yet have been inserted through side opening or port 61 or it may have been pre-inserted through side port or opening 61 but advanced only to a position within the second lumen 60 but not extending out of the second opening 54d in the distal end 55d. When treatment begins, the combining device 51, which in this example comprises an adjustable valve, may be set to allow flow only from the venous withdrawal cannula 58. Standard veno-arterial ECMO may then be performed wherein venous blood is drawn, by pump P, through the venous withdrawal cannula 58, through the flow controlling device 38, through inlet conduit 40 and into reservoir R. The pump P then further pumps the blood from reservoir R, through oxygenator O, through the first lumen 59 of aortic return/left ventricular venting cannula 55, out of the first opening 52 at the distal end 55d, and into the aorta AO. While this veno-arterial ECMO is proceeding, if it is determined that left ventricular venting is indicated, such as if high left ventricular afterload or evidence of left ventricular distention is observed, the left ventricular venting cannula 56 may then be advanced out of the second opening 54d in distal end 55d, advanced through the aorta AO, across aortic valve AV and into the left ventricle LV. With the distal portion 56d of the left ventricular venting cannula 56 positioned in the left ventricle LV, the combining device 51 may be set to combine flow from both the venous withdrawal cannula 58 and left ventricular venting cannula 56 at an initial ratio. This initial ratio may be dependent on a desired level of unloading of the left ventricle. The optional controller C may be programmed to receive signals from the sensor(s) 19 and, in response, to issue control signals to the combining device 51 (and possibly other elements of the ECMO device) to adjust the relative amount of left ventricular blood being withdrawn and combined with the concurrently withdrawn venous blood to maintain the sensed pressure or flow rate in the left ventricle below a particular limit or within a particular range.

### LV Unloading Shunt System Useable as Accessory to ECMO

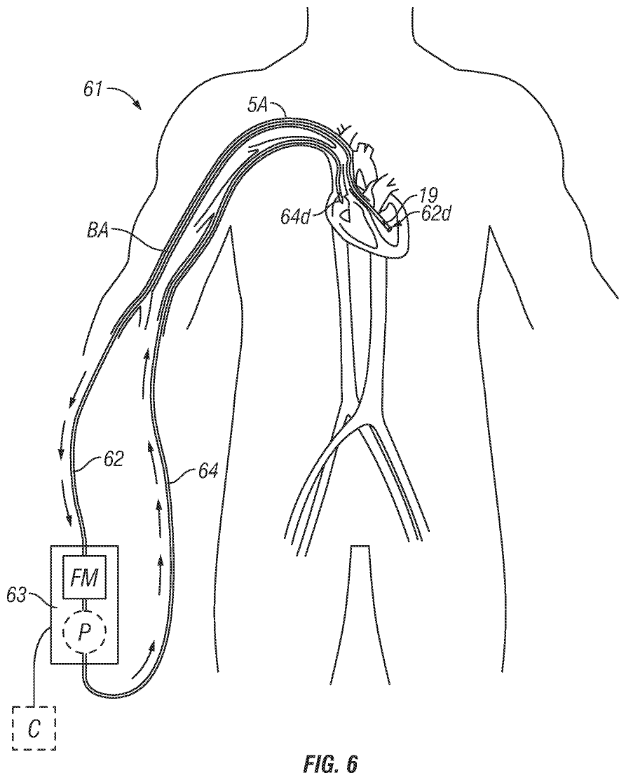

FIG. 6 shows a closed circuit left ventricle unloading shunt system 61 that is useable in conjunction with, but does not require connection to, a separate ECMO system. This shunt system may be used to lower and regulate pressure within the left ventricle LV. In this shunt system 61, a left ventricular unloading catheter 62 is inserted through a brachial artery BA, advanced through the arch of the aorta AO, across the aortic valve and to a position where its distal end 62d is within the left ventricle LV. Alternatively, the left ventricular unloading catheter 62 could be inserted into the left ventricle LV by any other suitable route, such as using a carotid artery or femoral artery insertion with transluminal advancement into the left ventricle LV. In the example shown, the left ventricular unloading catheter 62 has one or more optional flow and/or pressure sensor(s) 19 as described above located on its distal portion 62d.

A return cannula 64 is Inserted via a brachial vein and advanced to position where it's distal end 64d in within the superior vena cava or right atrium RA. Alternatively the return cannula 64 could be inserted into the vena cava or right atrium by any other suitable route, such as using a jugular or femoral vein insertion site with transluminal advancement to a caval or right atrial location.

The left ventricular unloading catheter 62 is connected to the inlet side of a device 63 comprising a pump P, flow meter FM and optional controller C. The return cannula 64 is connected to the outlet side of the device 63.

In operation, Pump P draws blood from the left ventricle LV, through left ventricular unloading catheter 62, through device 63, then though return catheter 64 and into the vena cava or right atrium. The pump P may be a variable speed pump or it may be equipped with a variable flow restrictor. The controller C may receive signals from the sensor(s) 19 and may be programmed to respond to those signals by adjusting the pump sped and/or flow restrictor to adjust blood flow through the shunt system as needed to maintain the sensed pressure or flow rate in the left ventricle below a particular limit or within a particular range.

Left Heart Unloading System Useable with Separate ECMO System

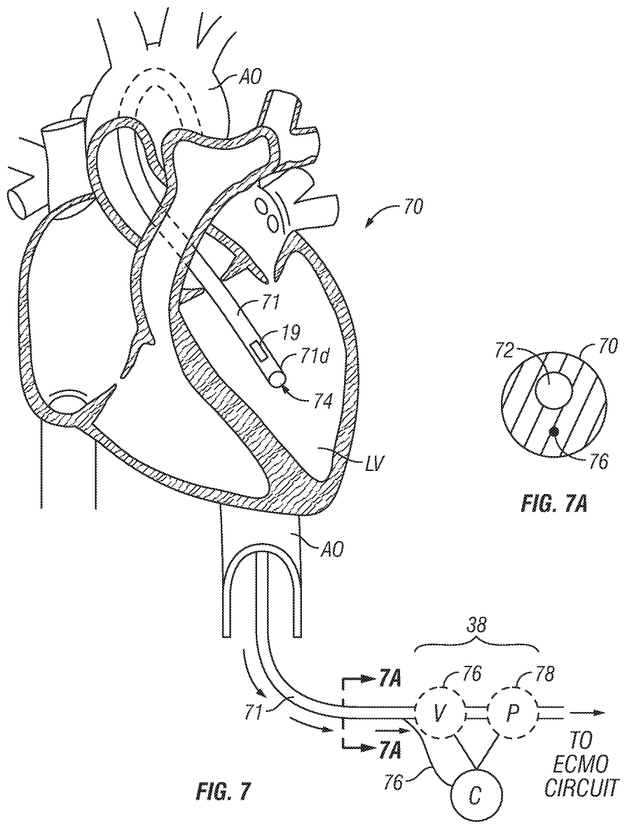

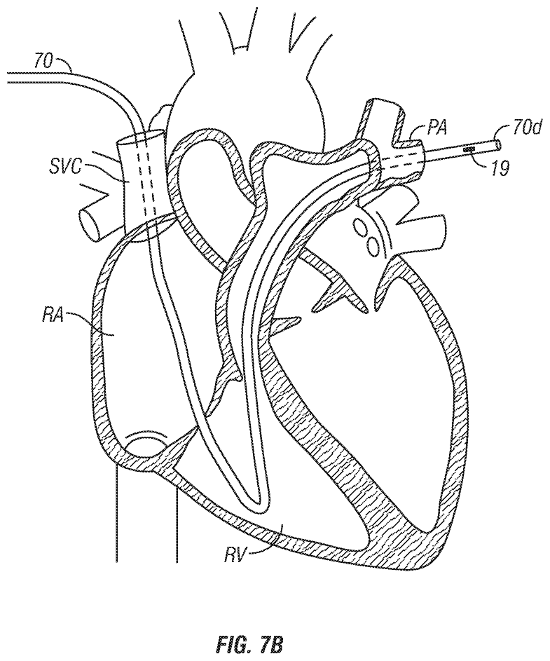

FIG. 7 through 7B show a pressure sensing and venting catheter system 70 that is useable for controlled reduction and maintenance of left ventricular pressure below a pressure limit or within a range set by a user. As noted above, the user-input pressure limit may be a value that is lower than the present arterial pressure so that LV pressure remains below the arterial pressure. By maintaining LV pressure below arterial pressure, during systole, the aortic valve will not open, the heart will not distend and left ventricular overload and its sequelae will be avoided.

In the example shown in FIG. 7A, a pressure sensing and drainage catheter 71 is inserted through the aorta AO and into the left ventricle LV. A at least one sensor 19 is positioned on the portion of the catheter 71 that resides within the left ventricle LV. A drainage lumen 72 extends through the catheter 71 and opens distally through an opening or port 74 at or near the distal end 71d of the catheter 71. A flow controlling device 38 such as a valve V and/or pump P is provided on a proximal portion of the catheter 71. The proximal end of the catheter is connected to an ECMO circuit or other extracorporeal system. In operation blood flows from the left ventricle LV. through opening or port 74, in the proximal direction through lumen 72 and into the ECMO circuit. The flow controlling device 38 controls the rate at which blood can pass through the lumen 72 of the catheter 71. For example, in a flow control device 39 comprising a valve V, the valve may have variable settings and/or in a flow controlling device 38 which comprises a pump P, the pump P may be operable at variable speeds to thereby control the rate at which blood is drawn through the lumen 72 of the catheter 71. The sensor(s) 19 communicate(s) with a controller C by way of one or more wire(s) 78 or alternatively by wireless connection.

In some embodiments, the sensor(s) 19 may comprise pressure sensor(s) which measure left ventricular pressure and communicate signals indicative of the measured left ventricular pressure to the controller C. The controller C may comprise or be connectable to a user interface, e.g., a display with touchscreen controls, which enables a user to input a desired left ventricular pressure limit to the controller C, e.g., via touchscreen or keyboard. Alternatively, the system may be programed to autoselect a value based on one or more sensed or derived physiological parameters, such as arterial blood pressure, peripheral vascular resistance. As the controller C receives signals from the sensor(s) 19 indicating the current left ventricular pressure, the controller, in accordance with its programming, then issues control signals to component(s) of the flow controlling device 38 in response to the signals received from the sensor(s) 19, to thereby cause blood to flow through the drainage lumen 72 at a rate which reduces or maintains the sensed left ventricular pressure below or within a permissible range of the user-input or autoselected pressure limit. In some embodiments of this system 70, the pump P of the flow controlling device 38 may be eliminated and the catheter 71 may be connected to the venous inlet and/or arterial outlet line of the ECMO system such that blood will be drawn through the catheter 71 by the separate ECMO pump or such that blood will merely drain (without pumping) through the catheter 17 and into the ECMO circuit while the valve V regulates the rate at which such drainage occurs.

In other embodiments, the sensor(s) may comprise apparatus for measuring a dimension or size (i.e., volume) of the left ventricle LV alone or in combination with left ventricular pressure. Sensors useable for measuring ventricular size include impedance or conductance based sensors. If the sensor(s) 19 measure only left ventricular dimension or size, the signals transmitted to the controller C will be indicative of left ventricular dimension or size and the controller C will be programmed to control the flow controlling device 38 in a manner that allows blood to drain from the left ventricle LV through drainage lumen 72 at a rate sufficient to prevent the measured dimension or size of the left ventricle from exceeding a preset or user-input limit. If the sensor(s) 19 measure both pressure and left ventricular size, the controller may be programmed to compute derivative values based on the combination of left ventricular pressure and size. For example, the controller may compute the size of the left ventricle LV and/or may generate a left ventricular pressure-volume loop and then issue control signals to the flow control device 38 based on the computed pressure-volume loop. One may use left ventricular catheters for measuring LV volume by conductance or both pressure and volume for purposes of computing left ventricular pressure-volume loops. Pressure and volume may be measured by means of a conductance catheter using previously reported techniques. See, for example: Baan, J, et al., Continuous Measurement of Left Ventricular Volume in Animals and Humans by Conductance Catheter, Circulation, 70:812-823 (1984); Steendijk, P., et al, Pressure-Volume Measurements by Conductance Catheter During Cardiac Resynchronization Therapy, European Heart Journal Supplements, 6 (Supplement D), D35-D42 (2004). In one example, the conductance catheter may have a plurality of electrodes at spaced-apart locations along a distal portion of the catheter that is positionable in the left ventricle. The electrodes are connected to a conditioning amplifier to measure conductance and convert it to volume. For example, eight (8) electrodes may be equally spaced in a longitudinal row on the distal portion of the catheter. The distal portion of the catheter is insertable into the left ventricle such that the electrodes are aligned generally along the axis of the left ventricle. The distance, L, between the electrodes may be selected so that the catheter is positionable such that electrode 1 is located at the apex of the left ventricle, electrode 8 is located in the aortic root just outside of the aortic valve and electrodes 2 through 7 are aligned between electrodes 1 and 8. The catheter may also include one or more lumen(s) or sensor(s) for pressure measurement within the left ventricle and/or aorta. A current of 20 kHz (0.07 mA root mean square) is applied between electrodes I and 8. The voltages measured between adjacent electrodes may have an order of magnitude of I mV root mean square. The system controller C or a separate processor/controller unit may be programmed to compute a time-varying left ventricular volume, V(t), on the measured conductance in the left ventricle. One non-limiting example of an equation that may be used is the following Equation 1:

V(t)=(1/α)(L²/σ_{b})G(t)−V_{c}

wherein α is a dimensionless constant, σ_{b} is the specific conductivity of blood measured by a calibrating cuvette, and G(t) is the sum of the conductances Gₙ(t) measured between the five pairs of adjacent electrodes:

$$G {( t )}_{n = 1}^{5} = \sum {G}_{( n )} t + 1 / 3 {G}_{l} ( t )$$

V_{c} is a correction term caused by the conductance, GP, of structures surrounding the ventricular cavity, calculated as follows:

V_{c}=(1/α)(L²/σ_{b})G^{p}

Pressure-volume loop data may be obtained by use of pressure-conductance catheters. These catheters may be 7French, over-the-wire, pigtail catheters of a type produced by several companies (e.g. CDLeycom, Zoetermeer, The Netherlands; Millar Instruments, Houston, Tex.). To generate the electric field, measure the resulting voltages, and acquire and handle the various signals, the catheter may be connected to dedicated equipment such as those available from Cardiac Function Lab CFL-512 or the Sigma 5 DF (CDLeycom, Zoetermeer, The Netherlands). Data analysis may be performed with software installed on the CFL-512 or by using other commercially available physiological data-analysis software, or software that is custom-made by various research groups. Certain conductance catheters and related apparatus useable for obtaining pressure-volume loop data are commercially available under the product names MPVS Ultra Pressure-Volume Loop System, PVR-1045 Pressure-Volume Catheter and Ventri-Cath Pig Catheter from Millar, Inc., 6001-A Gulf Freeway, Houston, Tex. 77023 USA.

In any embodiment, the controller C may include a user interface, such as a touch screen display or other type of display. In embodiments that utilize a pressure-volume loop, the pressure-volume loop or some graphic representation of the pressure-volume loop may be displayed. In embodiments where a pressure-volume loop is displayed, the display may be adapted to allow a user to input a desired pressure-volume loop size and the controller may be programmed to cause blood to drain at a rate that will cause the actual pressure-volume loop to approach or reach the desired size input by the user.

FIG. 7B shows an example of the system 70a being used with alternative placement of the pressure sensing and venting catheter 71 in the pulmonary artery PA rather than the left ventricle LV as shown in FIG. 7. In the example of FIG. 7B, the catheter 71 is inserted into the pulmonary artery PA using techniques known in the art. For example, the catheter 71 may be introduced into a vein such as the internal jugular, subclavian, or femoral vein and advanced into the right atrium RA, through the right ventricle RV and into the pulmonary artery PA. Proper movement of the catheter through the respective vessels and chambers of the heart as well as the intended placement in the pulmonary artery PA may be monitored and verified by observing pressure waveform changes using the pressure sensor 73 and/or by fluoroscopy or other suitable imaging.

The catheter 71 is placed in the pulmonary artery PA the system 70a is used in essentially the same manner as described above, except that the sensor(s) 19 will sense a pulmonary artery pressure and instead of removing blood directly from the left ventricle LV, the catheter will remove blood from the pulmonary artery PA, which in turn effectively lowers left ventricular pressure as desired. In some embodiments, the catheter 71 may be equipped with an occlusion balloon distal to the sensor(s) 19 and the pulmonary artery pressure measured may be pulmonary artery wedge pressure. The capillary wedge pressure is a measurement of the left ventricular pressure. The user-input target pressure will be an intended pulmonary artery pressure PAp, which may be a wedge pressure or other pulmonary artery pressure having known correspondence to a desired left ventricular pressure limit. In some embodiments of this system 70a, the catheter 71 may include, in addition to sensor(s) 19 for measuring a pulmonary artery pressure PAp, additional optional sensor(s) 19a positioned for measuring right ventricular pressure RVp. Signals from sensors 19 and 19a will be transmitted to the controller C and the controller C will be programmed to determine the difference between or ratio of pulmonary artery pressure PAp and right ventricular pressure RVp and to issue control signals to the flow controlling device 18 based on such difference or ratio.

Controlled Left Ventricular Unloading Using Pressure Measurements from Pulmonary Artery Catheter

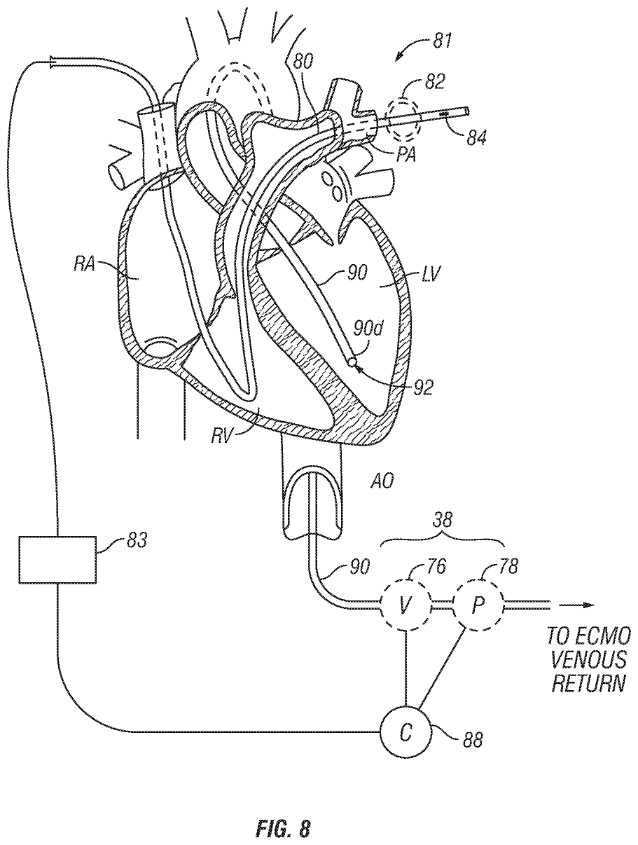

FIG. 8 shows an example of a system 81 wherein a left ventricular unloading catheter 90 is used in combination with a Swan Ganz pulmonary artery catheter 80, or other pulmonary artery catheter useable to measure pulmonary artery wedge pressure, to effect controlled unloading of the left ventricle LV in a patient who is being treated with ECMO. In this example, the drainage catheter 90 has a distal portion 90d that is advanceable through the aorta AO and into the left ventricle LV. As described above, this catheter 90 and any of the left ventricular catheters or cannulae described herein may have a pigtail or helical configuration and/or other curvate shape(s) to facilitate retrograde advancement through the arch or the aorta, across the aortic valve and into the left ventricle LV. A lumen 92 extends through the drainage catheter 90 and terminates distally at an opening or port in the distal end of the catheter 90. The pulmonary artery catheter 80 has a wedge balloon 82 and pressure sensor 84 which are useable to measure pulmonary artery wedge pressure. To obtain a measurement of pulmonary artery wedge pressure, the wedge balloon 82 is inflated to briefly cause full occlusion of the pulmonary artery. The pressure sensor 84 is located distal to the wedge balloon 82 and is useable to measure the pressure of blood in the pulmonary artery PA distal to the wedge balloon 82 while the wedge balloon 82 is fully occluding the pulmonary artery PS. This is known as Pulmonary Artery Wedge Pressure (PAP-W). In the absence of certain abnormal conditions, PAP-W can be viewed as an indicator of left ventricular end diastolic pressure. See, Falcov, R. E. et al.; Relationship of the Pulmonary Artery End-Diastolic Pressure to the Left Ventricular End-Diastolic and Mean Fling Pressures in Patients With and Without Left Ventricular Dysfunction Circulation; Circulation, Volume XLII, 65-73 (July 1970).

The left ventricular unloading catheter 90 is connected to the inlet side of a device 94 which comprises a valve 76 and/or pump 78 and a controller 88. The outlet side of that device 94 is connected to a ECMO circuit.

The pulmonary artery catheter 80 may be connected to a display device 83 which receives and displays pressures measured by the pressure sensor 84. When the wedge balloon 82 is inflated and PAP-W is measured, that PAP-W value may either be read from display device 83 and then manually entered into the controller 88 or the controller 88 may be programmed to acquire the PAP-W readings from the display device 83 by wired or wireless connection. The controller C is programmed to then control the valve 76 and/or pump 78 on the basis of the PAP-W readings obtained, to draw blood from the left ventricle LV through lumen 92 as needed to reduce or maintain the PAP-W at or below a set maximum value. Blood which is drained from the left ventricle LV through drainage catheter 90 will combine with blood flowing through the ECMO circuit, such as venous blood as it enters the ECMO circuit when V to A ECMO is being utilized.

### Systems Utilizing Pressure Feedback to Control LV Unloading

The examples described above include systems in which a pressure sensing device is placed in the left ventricle for directly sensing left ventricular pressure or in a pulmonary artery for sensing pulmonary artery wedge pressure as an estimation of or surrogate for left ventricular pressure. Signals from such pressure sensing devices may then be received by a controller which is programmed to use those signals as a basis for controlling a pump (e.g., the ECMO system pump or a separate pump) and/or valving apparatus to start, stop or adjust the rate at with blood is being removed from the left ventricle as needed to maintain a desired pressure or pressure range within the left ventricle. The following paragraphs describe additional pressure sensing systems and methods that may provide the desired signals (i.e., feedback) to the controller in any of the embodiments described herein.

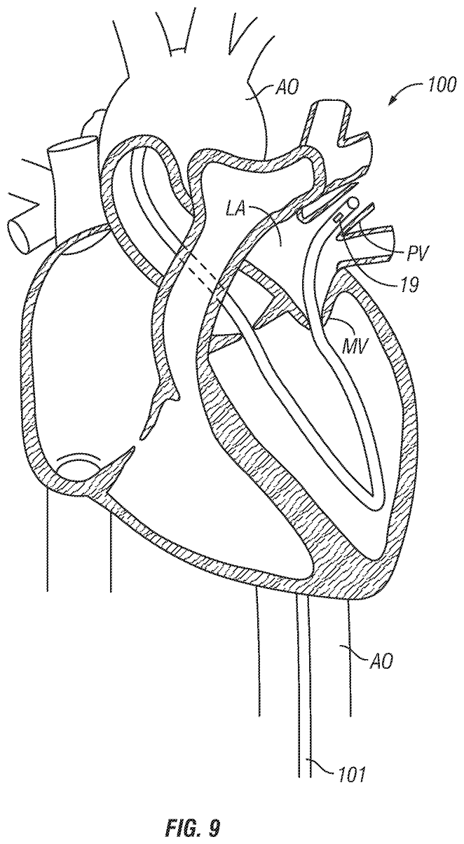

The examples described above include an embodiment wherein a drainage catheter 14 bearing a pressure sensor 19 is placed in the left ventricle LV, as shown in FIG. 4. In an alternative system 100 shown in FIG. 9, a pressure sensing and drainage catheter 101 may be placed in the pulmonary vein PV. This pressure sensing drainage catheter 101 has a pressure sensing device 19 that resides in a pulmonary vein PV and a lumen which connects to a venous or arterial line of the ECMO system in the same manner as described above in relation to FIG. 4. The pressure sensing drainage catheter 101 would be inserted into the arterial system, e.g., via femoral or carotid arteries, into the left ventricle, into the left atrium, and then into the pulmonary vein. Optionally, a suitable type of imaging may be used to facilitate placement of the catheter 24 into the pulmonary vein PV. One example of a method for retrograde, transaortic advancement of a catheter into the pulmonary vein PV is described by Sharif, A. Y. et al., Ablation of Focal Right Upper Pulmonary Vein Tachycardia Using Retrograde Aortic Approach, J Teh Univ Heart Ctr 3 146-149 (2010). The pulmonary venous pressure measured by the sensor(s) 19 on this catheter 101 is communicated by wired or wireless connection to the controller which then uses those signals to control the ECMO pump or a second pump 76 which differs from the ECMO pump, to withdraw blood from the pulmonary vein PV and through the drainage lumen of the catheter 101 to maintain the desired pressure within the pulmonary vein PV, which in turn maintains the desired pressure within the left ventricle

The pulmonary vein draining and pressure sensing catheter 101 may have a configuration similar to any of the catheters described herein. In an alternative, a pressure sensing and drainage catheter like the catheter 14, 101 shown in FIGS. 4 and 9 may be placed in the left ventricle LV or pulmonary vein PV and the drainage lumen of that catheter is connected to the venous or arterial line of the ECMO system without a pump such that blood drains from the lumen of the catheter into the venous or arterial line of the ECMO system Optionally, a flow controlling device 38 (e.g., a pump and/or valving/flow restricting device) as described above may be controlled by a controller based on the pulmonary vein pressure being measured by the sensing device 19 to maintain the sensed pressure or flow rate in the pulmonary vein or left ventricle below a particular limit or within a particular range.

In another alternative, a catheter having a wedge balloon (e.g., a Swan-Ganz catheter) is placed in the pulmonary artery and its balloon is inflated to measure pressure distal to the balloon, providing a measurement of the LV pressure. This catheter connects to and pumps into the venous return of the ECMO system. Based on the measured pressure, the pump (e.g., a second pump that differs from the ECMO pump) may be controlled to decrease or increase the blood flow rate to hold the pressure at a preset value.

In another alternative, a pressure sensing and drainage catheter like the catheter 14, 101 shown in FIGS. 4 and 9 may be placed in the left ventricle LV or pulmonary vein PV and the drainage lumen of that catheter may be connected to the venous return of the ECMO system. This catheter connects to and drains (e.g., without a pump) into the venous return of the ECMO system. A catheter (e.g., Swan Ganz) is placed into the pulmonary capillary system and the balloon on the catheter is inflated and pressure distal to the balloon is measured, providing a measurement of the LV pressure. Based on the measured pressure, a control system may control a valve, by opening or closing the valve, to modify the blood flow rate to hold the pressure at a preset value. Such a system may not require a second separate pump other than the ECMO pump.

In another alternative, pressure sensing devices may be placed at separate locations and the system may operate to maintain a desired difference or delta between pressures sensed at those two locations. For example, one pressure sensing device may be placed in the left ventricle and a second pressure sending device may be placed in the aorta. Signals from both pressure sensing devices may be communicated to the controller and the controller may be programmed to control the amount of blood removed from the left ventricle LV to maintain a desired pressure differential between those two locations as set by a user. For example, as explained in detail above, the controller may be programmed to maintain the left ventricular pressure sufficiently below the aortic pressure to prevent opening of the aortic valve and expulsion of blood from the left ventricle into the aorta.

In one such embodiment, a drainage catheter with two pressure sensors is placed into the left ventricle or pulmonary vein. A first sensor is on or near the catheter distal tip and becomes positioned in the left ventricle LV or pulmonary vein PV. A second sensor is proximal to the first sensor and positioned in the aorta. Signals from both sensors are communicated to the controller which is programmed to use those signals to monitor the difference in pressure between first sensor (in the Left Ventricle LV or pulmonary vein PV) and the second sensor (in the aorta AO). Based on that measured pressure difference, the controller then controls a pump or valving device to maintain a desired pressure difference that is preset or set by a user. The goal is to keep the left ventricular pressure below the arterial pressure, thereby keeping the heart relaxed and less distended during ECMO where the pressure is high.

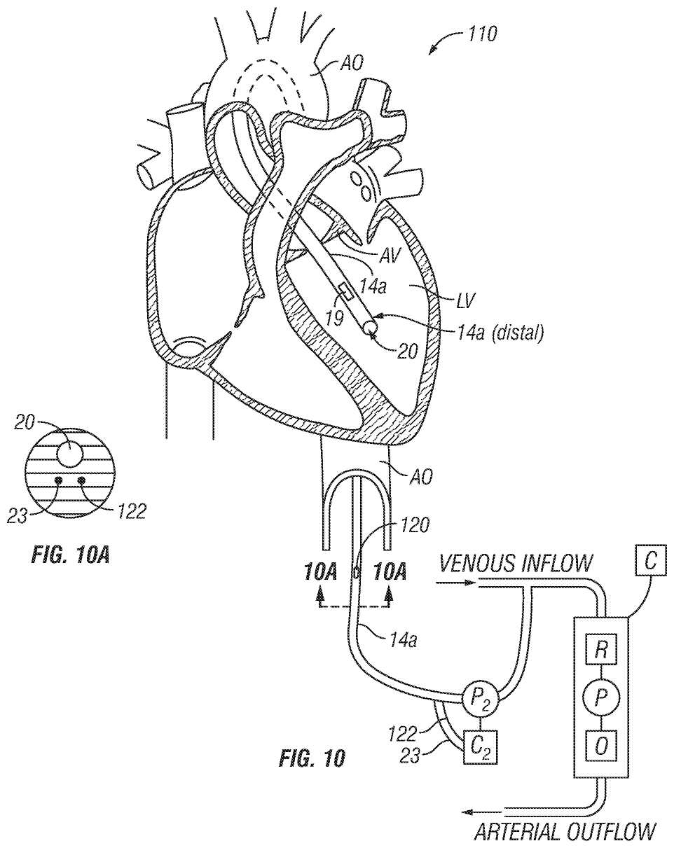

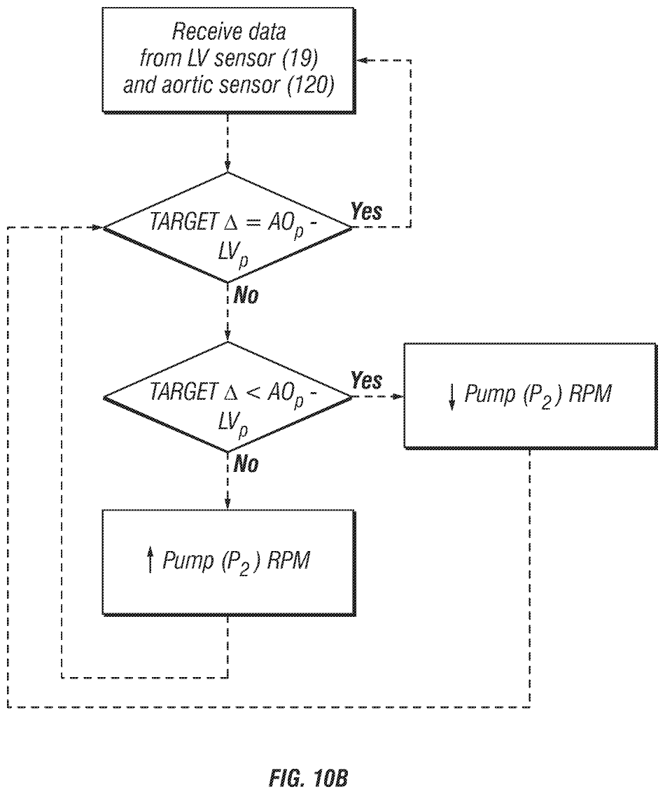

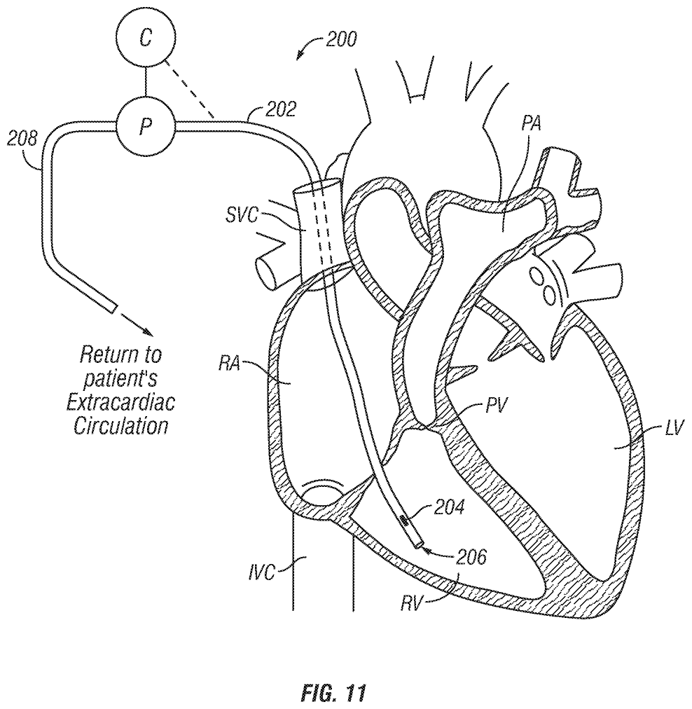

FIGS. 10 and 10A show a non-limiting example of a left heart unloading system 110 wherein the difference between left ventricular pressure and aortic pressure is used as the basis for controlling the amount of blood being removed from the left ventricle LV. This system comprises a left ventricular drainage/pressure sensing catheter 14a which has a drainage lumen 20 and left ventricular pressure sensor 19 as described above in relation the embodiment of FIG. 3. Additionally, this catheter 14a also has an aortic pressure sensor 120 positioned on a portion of the catheter that resides in the aorta AO. This system further includes a pump P₂ for pumping blood in a proximal direction through the drainage lumen 20 and a pump controller C₂ for controlling the pump P₂. A proximal end of the catheter 14a is connected to the venous inflow conduit of an ECMO system of the type described above. The left ventricular pressure sensor is connected by wire 23, or by wireless connection, to the pump controller C₂. Also, the aortic pressure sensor 120 is connected by wire 122, or by wireless connection, to the pump controller C₂. The pump controller C₂ receives signals from the left ventricular sensor 19 and aortic sensor 120 representing pressure in the left ventricle and aorta, respectively.

As shown in the diagram of FIG. 10B, the pump controller C₂ is programmed to process those received signals to periodically or continually determine the difference between a left ventricular pressure LV, (e.g., mean left ventricular pressure, peak left ventricular pressure, etc.) and an aortic pressure AO, (e.g., mean aortic pressure, peak aortic pressure, etc.). The pump controller C₂ may be preprogrammed at the time of manufacture with standard acceptable value(s) or range(s) for this difference or, alternatively, the pump controller C₂ may be equipped with a user interface and programmed to receive, via such user interface, user input of acceptable value(s) or range(s) for this difference. Thereafter, so long as the measured difference AOₚ−LVₚ is equal to an acceptable value or within an acceptable range (i.e., a target delta), the pump controller C₂ will make no change to the current operation or nonoperation of the pump P₂. However, if the measured difference AOₚ−LVₚ becomes unequal to an acceptable value or outside of an acceptable range (i.e., a target delta), the pump controller will issue control signals to cause the pump to start, stop, speed up or slow in accordance with programming of the pump controller aimed at maintaining the measured pressure difference AOₚ−LVₚ equal to a target delta. In a typical application, the acceptable range may be any measured AOₚ−LVₚ greater than 0, but not greater than the arterial pressure, thereby keeping the left ventricular pressure sufficiently below the aortic pressure to allow the ventricle to relax. For example, the left ventricular pressure may be sufficiently low to prevent opening of the aortic valve and ejection of blood from the left ventricle LV into the aorta AO.

FIG. 11 shows a non-limiting example of a left heart unloading system 200 wherein the difference between right ventricular pressure and pulmonary artery pressure is used as the basis for controlling the amount of blood being removed from the pulmonary artery PA, thereby decreasing the amount of blood that enters the left ventricle LV.

In another embodiment, aortic pressure may be used as the basis for controlling the amount of blood being removed from the left ventricle LV. For example, two pressure sensors may be positioned in two different locations within the aorta, e.g., using one or more catheters having one or more pressure sensors located thereon. A first sensor may be located in the aortic arch and second sensor may be located in some lower portion of the ascending or descending aorta. A variety of metrics may be derived from the pressure sensor readings, such as pulse wave transit time or pulse wave velocity, from which afterload may be derived, from which the amount of work performed by the LV may be derived.

### Right Heart Unloading

Dysfunction of the right ventricle RV can arise from a number of clinical conditions including pulmonary arterial hypertension, chronic pressure overload, cardiomyopathies, pulmonary valvular stenosis, arrhythmias and sepsis. Right ventricular disfunction has been reported to be the main cause of mortality in patients suffering from pulmonary artery hypertension. Simon, M. A., et al., Right Ventricular Dysfunction and Failure in Chronic Pressure Overload, Cardiology Research and Practice, Vol. 2011, Article ID 568095, 7 pages (2011). Moreover, the right and left ventricles are interdependent and, therefore, volume or pressure overloading of the right ventricle RV can also affect function of the left ventricle LV. Naeige, R. et al., The Overloaded Right Heart and Ventricular Interdependence; Cardiovasc Res., 1; 113(12), pages 1474-1485 (2017). Thus, in at least some cases, unloading of the right heart may be of therapeutic benefit irrespective of whether the subject's blood is also being circulated through a separate ECMO device or extracorporeal support device as described above in relation to left ventricular unloading. Accordingly, in addition to their use in unloading the left ventricle LV as described above, where feasible, any system described herein may also be useable for therapeutic unloading of the right heart (e.g., venting blood from the right ventricle or pulmonary artery) thereby decreasing overload of the right ventricle RV and reducing pulmonary artery pressure. Such systems may be useable to treat right ventricular disfunction in patient's suffering from pulmonary artery hypertension and/or other disorders, some of which are listed above. In certain embodiments, unloading of the right heart may also provide a therapeutic benefit to the left heart and improve left heart function.

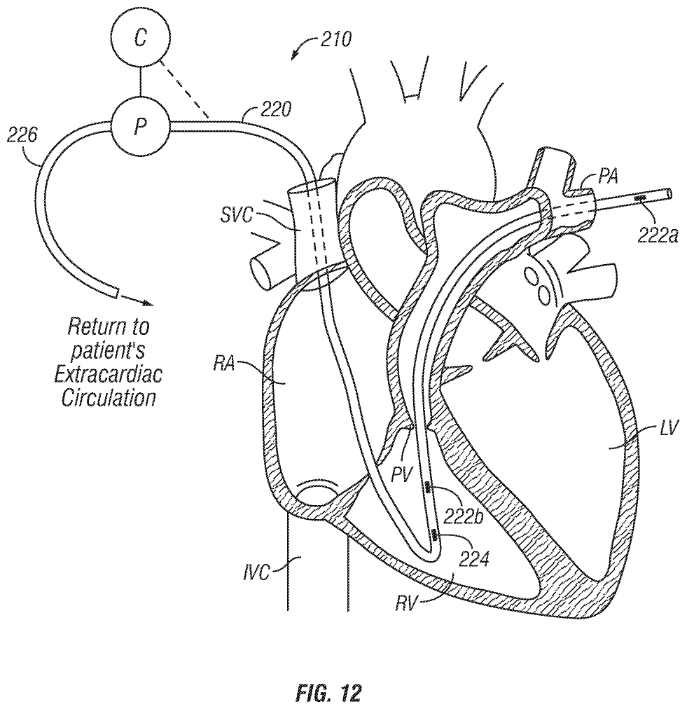

FIGS. 11 and 12 show non-limiting examples of systems that may be used to effect unloading of the right heart.

Referring specifically to FIG. 11, there is shown a system 200 wherein a drainage conduit 202 is advanced into the right ventricle RV. At least one sensor 204 is used to sense a variable that is indicative of right ventricular overload (e.g., RV pressure, RV volume, RV size, RV wall thickness, etc.). A pump P is connected to the drainage conduit 202 to pump blood from the right ventricle RV through drainage lumen 206 which extends through drainage conduit 202. As described above, the sensor(s) 204 are connected by wired or wireless connection to controller C. Controller C is programmed to control the pump P, in response to signals received from the sensor(s) 204, thereby removing blood from the right ventricle at a rate or in an amount that is effective to avoid or reverse overload of the right heart. Blood which drains from the right ventricle RV through drainage conduit 202 is returned to the subject through return conduit 208. Return conduit 208 may infuse blood directly into the subject's vasculature or, if the subject is connected to another extracorporeal device though which the subject's blood is being circulated, the return conduit 208 may be connected to that other extracorporeal device so that blood from the return conduit 208 becomes combined with blood circulating through the other extracorporeal device. Examples of such other extracorporeal devices include but are not necessarily limited to; an ECMO device, a cardiopulmonary bypass device or an extracorporeal circulatory support or cardiac assist device.

In the example of FIG. 11, the drainage cannula 202 is advanced only into the right ventricle RV and no navigation of the drainage cannula 202 across the pulmonary valve PV and/or into the pulmonary artery PA is required, thereby allowing relatively simple and rapid placement of the drainage conduit 202. Also, in the example of FIG. 11, the sensor(s) 204 is/are positioned within the right ventricle RV and may be used for monitoring right ventricular variables such as: pressure within the right ventricle or an indication or right ventricular volume such as a pressure volume loop indicating pressure/volume within the right ventricle.

FIG. 12 shows another embodiment of a system 210 for right heart unloading. In this system 210, a drainage catheter 220 is advanced through the right ventricle RV, across the pulmonary valve PV, and into a pulmonary artery PA. One or more sensor(s) 222a is/are positioned on the portion of the drainage conduit 220 that becomes positioned in a pulmonary artery and such sensor(s) is/are useable to sense a variable within the pulmonary artery that is indicative of right heart overload and/or pulmonary artery hypertension. In some embodiments, the drainage conduit 220 may be equipped with an optional wedge balloon as shown in FIG. 7C and discussed above. Examples of variables that may be sensed within a pulmonary artery to assess the existence of or potential for right heart overload include, for example, pulmonary artery pressure, pulmonary artery wedge pressure, pulse transmission rate within pulmonary artery and indicators of pressure induced dilation or enlargement of the pulmonary artery such as a pressure-volume loop.

Alternatively or additionally, in some embodiments of the system 210, one or more additional sensor(s) 222b may be positioned at location(s) outside of the pulmonary artery PA, such as in the right ventricle RV. If, as shown in FIG. 12, the drainage conduit 220 is equipped with both sensor(s) 222a and 222b, such sensors 222a and 22b may be used in combination with the controller C to compute differences in pressure between the right ventricle (sensor 222b) and pulmonary artery PA (sensor 222b). In any event, signals from sensor(s) 222a and/or 222b (if present) are received and used by controller C to control the rate at which pump P pumps blood through the drainage conduit 220, thereby unloading the right heart and reducing pulmonary congestion.

In the example shown in FIG. 12, the drainage conduit 220 has a drainage lumen 224 which terminates distally in a side wall opening on a portion of the cannula 220 that resides within the right ventricle RV when the cannula 220 is inserted and, thus, blood drains directly from the right ventricle and through the drainage lumen 224 of drainage cannula 220. However, additionally or alternatively, in some embodiments, the drainage lumen 224 may extend to opening(s) at the distal end of the cannula 22 (see embodiment of FIG. 11) or at other location(s) on a portion of the cannula 220 that resides in a pulmonary artery PA when the cannula 220 is inserted and, in such embodiments. blood may be drained from the pulmonary artery instead of, or in addition to, drainage of blood from the right ventricle RV. Blood which is drained from the right ventricle RV and/or pulmonary artery PA through drainage conduit 222 is then returned to the subject through return conduit 226.

As explained in relation to FIG. 11, the return conduit 226 may infuse blood directly into the subject's vasculature or, if the subject is connected to another extracorporeal device (examples of which are provided above) the return conduit 226 may be connected to that other extracorporeal device so that blood from the return conduit 226 becomes combined with blood circulating through the other extracorporeal device. Where feasible in any of the herein-described embodiments, sensor(s) 19, 204, 222a, 222b and the controller C may be operative/programed to sense and compute a variable indicating whether unloading of a chamber of the heart is desirable or indicated based on a rate of pulse transmission or other pressure-volume or flow-volume relationship. For example, left ventricular afterload may be estimated or determined based on pressure-flow relations, such as pulse transmission rate, thereby allowing for quantification of various components of left ventricular load using biomechanical models of the hemodynamics. See, for example, Chirinos, J. A., et al., Noninvasive Evaluation of Left Ventricular Afterload Part 2: Arterial Pressure-Flow and Pressure-Volume Relations in Humans; Hypertension, 56:563-570 (2010). To obtain such measurements pressure and flow sensors may be placed at locations in the subject's aorta. Data from those sensors is then transmitted to the system controller C, or a separate processor, which is programmed to use such data as a basis for quantifying various components of left ventricular afterload.

Essentially, wave reflections typically arrive at the proximal aorta in mid-to-late systole, with superimposition of reflected and incident waves. It may be assumed that measured pressure equals the sum of forward and backward pressure and measured flow equals the sum of forward and backward flow. If the slope of the pressure-flow relationship in the absence of wave reflections (i.e., Zc) is known, a relationship between pressure and flow waves can be determined and differences in their respective waveforms can be used to ascertain the forward (Pf) and reflected (backward, Pb) components thereof. This procedure is commonly called wave separation analysis and can be mathematically described as follows:

Pf=(P+Q*Zc)/2

Pb=(P−Q*Zc)/2

In any embodiment described herein, were a difference between two pressures is calculated such difference may be based on mean pressures (e.g., mean left ventricular pressure vs. mean arterial pressure) or may be based on pressures taken at specific points of a pressure waveform (e.g., left ventricular end diastolic pressure versus the corresponding pressure within the aorta at the same time point).

It is to be appreciated that, although the disclosure has been described hereabove with reference to certain examples or embodiments of the disclosure, various additions, deletions, alterations and modifications may be made to those described examples and embodiments without departing from the intended spirit and scope of the disclosure. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any disclosure or example described herein may optionally exist or be utilized in the absence or substantial absence of any other element, step, member, component, composition, reactant, part or portion unless otherwise noted. All reasonable additions, deletions, modifications and alterations are to be considered equivalents of the described examples and embodiments and are to be included within the scope of the following claims.

1. A system for delivering blood to a subject, said system comprising:
an extracorporeal pulmonary and/or circulatory support system which comprises:
a blood inlet, a blood pump, and a blood outlet;
a venous blood withdrawal cannula insertable into venous vasculature of the subject and connectable to the blood inlet; and
a blood return cannula insertable into arterial vasculature of the subject and connectable to the blood outlet;
a drainage conduit having:
a distal portion configured for insertion into a left ventricle, a pulmonary vein, or a pulmonary artery of the subject; and
a proximal end connectable to the extracorporeal system such that blood may flow from the left ventricle, the pulmonary vein, or the pulmonary artery, through the drainage conduit, and into the extracorporeal system such that the blood combines with other blood circulating through the extracorporeal system;
at least one sensor for sensing at least one parameter that is indicative of a left ventricular pressure, a left ventricular size, or a left ventricular dimension, or indicative of a hemodynamic condition which is associated with a left ventricular overload or an incomplete left ventricular emptying;
a flow-controlling device for starting, stopping or controlling a rate of blood flow from the left ventricle, through the drainage conduit, and into the extracorporeal system to combine with the other blood circulating through the extracorporeal system; and
a controller configured to:
receive signals from that least one sensor, and
respond to the signals by causing the flow-controlling device to start, stop, or change the rate of blood flow from the left ventricle, the pulmonary vein, or the pulmonary artery, through the drainage conduit, and into the extracorporeal system.

2. A system according to claim 1 wherein the extracorporeal system further comprises a blood oxygenator for oxygenating blood circulating through the system.

3. A system according to claim 1 further comprising a reservoir.

4. A system according to claim 1 wherein said at least one parameter or variable is selected from a left ventricular pressure, a left ventricular end-diastolic pressure, a left ventricular end-systolic pressure, a left ventricular end-diastolic volume, a left ventricular end-systolic volume, a pulmonary wedge pressure, a cardiac afterload, a pulmonary vein pressure, and a difference between the left ventricular pressure and an extracardiac arterial pressure.

5. A system according to claim 1 wherein said at least one parameter or variable is a left ventricular pressure, the drainage conduit is placed in the left ventricle and said at least one sensor comprises a pressure sensor located on the drainage conduit so as to directly measure said left ventricular pressure.

6. A system according to claim 1 wherein the at least one parameter includes a pulmonary wedge pressure, wherein the drainage conduit is placed in a pulmonary vasculature, and where the at least one sensor comprises a pressure sensor and a balloon useable in combination to measure the pulmonary wedge pressure.

7. A system according to claim 1 wherein said at least one parameter or variable is a pulmonary venous pressure, the drainage conduit is placed in a pulmonary vein and said at least one sensor comprises a pressure sensor located on the drainage conduit so as to measure said pulmonary venous pressure.

8. A system according to claim 1 wherein the at least one parameter includes a difference between a first pressure measured at a first location selected from the left ventricle, the pulmonary vein, or the pulmonary artery, and a second location in an aorta or extracardiac arterial vasculature, wherein the drainage conduit is placed at the first location and the at least one sensor comprises a first pressure sensor for sensing a first pressure at the first location and a second sensor for sensing a second pressure at the second location.

9. A system according to claim 6 wherein:
the sensor is located on a pulmonary artery wedge pressure measuring catheter and is useable to measure pulmonary artery wedge pressure; and
the controller receives signals from the sensor indicating pulmonary artery wedge pressure and is further programmed to correlate pulmonary artery wedge pressure to left ventricular pressure.

10. A system according to claim 1 wherein the flow-controlling device comprises a valving device which controls an amount of the blood that flows through the drainage conduit.

11. A system according to claim 10 wherein the blood pump of the extracorporeal system draws the blood through both the venous blood withdrawal cannula and the drainage conduit, and wherein the valving device varies relative amounts of the blood being drawn through the venous blood withdrawal cannula and the drainage conduit.

12. A system according to claim 1 wherein the flow-controlling device comprises a pump which controls the rate of blood flow through the drainage conduit.

13. A system according to claim 12 wherein the pump is separate from the blood pump of the extracorporeal system.

14. A system according to claim 12 wherein the pump is a pump within the extracorporeal system.

15. A system according to claim 1 wherein the flow-controlling device comprises a valving device and a pump, in combination.

16. A system according to claim 1 wherein the drainage conduit comprises a first lumen of a catheter device, and wherein the blood return cannula comprises a second lumen of that same catheter device.

17. A system according to claim 16 wherein the catheter device comprises:
a first tube having a first lumen which functions as the drainage conduit; and
a second tube having a second lumen which functions as the blood return cannula, the second tube being initially positioned within an aorta, and the first tube being subsequently advanceable from the second tube, through the aorta, across an aortic valve and into the left ventricle.

18. A system according to claim 17 wherein the second tube includes a utility lumen in addition to said second lumen and wherein the first tube is advanceable through the utility lumen of the second tube.

19. A system according to claim 16 wherein the first lumen communicates with a left ventricular blood withdrawal opening or port located in a distal portion of the catheter device, and wherein the second lumen communicates with a blood infusion opening or port located on the catheter device proximal to the left ventricular blood withdrawal opening or port

20. A system according to claim 16 wherein the first lumen is connectable to the blood inlet of the extracorporeal system, and wherein the second lumen is connectable to the blood outlet of the extracorporeal system.

21.-65. (canceled)
